(19) United States
(12) Patent Application Publication
(10) Pub. No.: US 2010/0112549 A1
(43) Pub. Date: May 6, 2010

(54) Microorganism Detection Method and Apparatus

(75) Inventors: Diego Rey, Palo Alto, CA (US); Carl Batt, Groton, NY (US); Leonardo Maestri Texeira, Ithaca, NY (US)

Correspondence Address: BOND, SCHOENECK & KING, PLLC, 10 BROWN ROAD, SUITE 201, ITHACA, NY, 14850-1248, US

(73) Assignee: CORNELL UNIVERSITY, Ithaca, NY (US)

(21) Appl. No.: 12/595,580

(22) PCT Filed: Apr. 18, 2008
(86) PCT No.: PCT/US08/60836

§ 371 Date: Oct. 12, 2009

Related U.S. Application Data
(60) Provisional application No. 60/912,553, filed on Apr. 18, 2007.

(51) Int. Cl. C12Q 1/70; C12N 7/01

(52) U.S. Cl. 435/5; 435/235.1

(57) ABSTRACT

Embodiments of the present invention relate to selective organism detection, and, more particularly to recombinant bacteriophages and the use of such recombinant bacteriophages to detect target bacteria and to detect specific nucleic acid sequences within said target bacteria thus allowing for the detection of phenotypic characteristics of said bacteria such as determining drug(s) to which such target bacteria are resistant. The present invention further relates to sample preparation apparatuses for preparing samples for detection and analysis using bacteriophage-based techniques, that are low in cost, easy to use, and do not require technical expertise or any additional laboratory infrastructure to perform.

### CROSS-REFERENCE TO RELATED APPLICATIONS

This application claims priority to U.S. Provisional Patent Application Ser. No. 60/912,553, filed Apr. 18, 2007, the entirety of which is hereby incorporated by reference herein.

### BACKGROUND OF THE INVENTION

1. Field of the Invention

Embodiments of the present invention relate to selective organism detection and, more particularly, to recombinant bacteriophages and the use of such recombinant bacteriophages to detect target bacteria and to determine drug(s) to which such target bacteria are resistant.

2. Description of the Related Art

The ability to identify specific types of bacteria is of great importance to health-care providers, farmers, and ultimately patients and consumers around the world. Illustrative of this fact, is that the annual worldwide bacterial in vitro diagnosis market is about (USD) $10 billion. Further, there are many different specific (but not exhaustive) illustrative examples that highlight of need for specific, sensitive, accurate, and reproducible bacterial diagnostics (products, kits, and methods), as discussed below.

For example, bovine mastitis, an infection caused by bacterial cells, results in the inflammation of the bovine breast, reduction in milk yield and a decrease in milk quality. This condition is caused by the bacteria Staphylococcus aureus and Staphylococcus agalactiae. This reduction in milk yields and quality in the western world alone cause annual financial losses estimated at $3.7 billion.

Bovine tuberculosis (Mycobacterium bovis), is another example of a bacteria that causes great financial loses worldwide. In 2005, for example, 12 of a heard of 55 cattle in a small Michigan farm tested positive for bovine tuberculosis. The farm was forced to destroy the entire herd of cattle, along with an entire herd of hogs. Tuberculosis testing in cattle requires the animal to be held for 2 days, and tests are false positive 5 percent of the time. Often entire herds have to be quarantined or destroyed. The annual worldwide financial losses are estimated at (USD) $3 billion. Moreover, M. bovis can infect humans.

Bacterial food borne diseases pose a significant threat to human health in causing ˜76 million illnesses, 325,000 hospitalizations, and 5,000 deaths in the US annually. Economic losses in the US due to insects and microbes are estimated between $5 and $17 billion annually.

For example, in 1996, juice that was contaminated with the bacteria Escherichia coli was released into the public by juice maker Odwalla which resulted in one death and 66 illnesses. The company paid a $1.5 million fine, and the recall alone cost the company $6.5 million. In 2006, an E. coli O157:H7 outbreak from contaminated Dole brand spinach originating from California resulted in 205 illnesses and 3 deaths.

Tuberculosis is a leading cause of death worldwide. One third of the world's population is infected with Mycobacterium tuberculosis, the bacterium that causes tuberculosis. Every day 25,000 people are infected and 5,000 people die from the disease. Furthermore, due primarily to poor diagnosis, multidrug resistant strains of M. tuberculosis are emerging and the reemergence of tuberculosis as a worldwide epidemic has become a real threat. The worldwide annual market for tuberculosis diagnostics is $1.8 billion.

According to leading international health organizations such as the World Health Organization (WHO), there is an essential need for quicker and more reliable diagnostics that can be feasibly implemented in developing countries that have little infrastructure and require low-cost diagnosis options. For example, India is the most afflicted country by tuberculosis, with 1.79 million new cases emerging annually and with only 46% of total infections actually detected due to poor bacterial diagnosis options available.

MRSA is a drug-resistant version of the common Staphylococcus aureus bacteria and is carried by 2.5 million people in the US. A carrier can be a healthy individual, and still be highly contagious, due to the nature of the MRSA bacterium. The bacteria are highly contagious and spread by touch. Approximately 86% of all infections occur within hospitals, and these infections carry a 20% mortality rate. This bacterium costs an average of $21,000 over the standard costs to treat, and kills approximately 19,000 people in the US annually.

There are several target areas where bacterial detection/diagnosis is advantageous including: environmental samples, plant samples, veterinary samples, food samples, livestock samples, and medical samples. These target areas/samples can be derived from many different sources including: environmental samples can be derived from sources such as water (e.g. rivers, lakes, ponds, oceans) the atmosphere, soil, mineral, as well as from surfaces and natural and synthetic materials; plant samples can be derived from sources such as live or dead natural vegetation, crops; veterinary samples can be derived from sources such as live or dead household animals, farm animals, and wild animals; food samples can be derived from sources such as any natural or synthetic food product intended for human, animal, or plant use; livestock samples can be derived from sources such as animals intended for human consumption; and medical samples can be derived from sources such as human tissue, blood, and sputum, and other bodily fluids.

A bacteriophage or phage is defined as a virus that infects bacteria. Bacteriophages have a high specificity to their corresponding host bacteria. To infect bacteria, the bacteriophage attaches to specific receptors on the surface of the bacteria. This attachment determines the host range of each bacteriophage, and normally is restricted to some genera, species, or even subspecies of bacteria. This bacteriophage specificity could provide clinicians, laboratory technicians, technicians in the field, as well as consumers, with the ability to identify (detect or diagnose) specific types of bacteria by exploiting this bacteriophage characteristic.

Bacteriophages experience two types of natural life cycles, or methods of viral reproduction, known as the lytic cycle and the lysogenic cycle. In the lytic cycle, host cells will be broken and suffer death after replication of the virion. In contrast, the lysogenic cycle does not result in immediate lysing of the host cell and consequential host cell death; rather, the bacteriophage genome integrates with the host DNA, or establishes itself as a plasmid, and replicates along with the organism's genome. The endogenous bacteriophage remains dormant until the host is exposed to specific conditions (e.g., stress) at which point the bacteriophage may be activated, initiating the reproductive cycle resulting in the lysis of the host cell.

There are conventional methods that exploit the specificity of the bacteriophage/bacteria interaction, which typically explore the lytic cycle of the bacteriophage. In fewer cases, these conventional methods explore the lysogenic cycle. Typically, as discussed in the reported literature, a reporter gene is incorporated into the host indiscriminately after the initial bacteriophage-host interaction takes place or a reporter molecule is fused to a phage and the amplification of this phage or the expression of the reporter molecule is detected after the recombinant bacteriophage infects its host bacteria. In some previous works, the use of bacteria-specific promoters and/or origin of replication are discussed in order to increase the specificity of the detection method. Furthermore, reporter genes are disclosed that express enzymes (e.g. luciferase, or beta galactosidase) which require indirect detection through the addition of other reporter bacteria or substrates.

Detection and analysis of bacteria from samples suffers unless steps are taken to prepare the samples for examination. This preparation can involve physical and chemical manipulations of the sample in order to improve the efficacy of a diagnosis. These steps are often taken in order to remove factors from the sample that may inhibit a detection methodology. Another predominant use of sample preparation is for concentrating the bacteria within the sample in order to improve the likelihood of detection as well as the robustness of the data that can be gathered from the bacteria in the sample.

Two specific examples where sample preparation has been addressed for clinical diagnosis of Mycobacterium tuberculosis are highlighted as follows. In the most comprehensive review of sample preparation of clinical sputum samples for detection of tuberculosis via smear microscopy to date (see Karen R Steingart, V. N., Megan Henry, Philip C Hopewell, Andrew Ramsay, Jane Cunningham, Richard Urbanczik, Mark D Perkins, and M. P. Mohamed Abdel Aziz, Sputum processing methods to improve the sensitivity of smear microscopy for tuberculosis: a systematic review. Lancet Infectious Diseases, 2006. 6: p. 664-674, which is hereby incorporated by reference herein in its entirety), the results showed that the concentration of the bacteria by centrifugation and any kind of conventional chemical processing of the sputum improved the sensitivity for detection. In another study looking specifically at processing sputum for detection of tuberculosis using a bacteriophage-based method (see D. J. Park, F. A. D., A. Meyer, and S. M. Wilson, Use of a Phage-Based Assay for Phenotypic Detection of Mycobacteria Directly from Sputum. Journal of Clinical Microbiology, 2002. 41(2): p. 680-688 which is hereby incorporated by reference herein in its entirety), both chemical and physical processes were determined to significantly improve the yield of the technique by chemically removing a factor in the sputum that inhibits bacteriophage infection and by concentrating the sample via centrifugation.

The conventional techniques for employing sample preparation require significant expertise and laboratory infrastructure for implementation. In addition, sample preparation often introduces a greater risk of contamination, to the samples hindering diagnosis, and also to clinical staff posing a higher risk for contracting disease.

Sample preparation of sputum for diagnosis of tuberculosis, for example, requires several steps of processing with chemicals and centrifugation. Several tubes are used in the process and a laboratory centrifuge is required.

Examples of sample preparation kits include the MYCOPROSAFE® sample preparation kit produced by Salburis, Inc. (Woburn, Mass.), and the FASTPlaque-Response™ tuberculosis diagnostic kit produced by Biotec Laboratories, LTD (Suffolk, England). The MYCOPROSAFE® sample preparation kit is used for processing sputum for tuberculosis diagnosis. Although this kit provides the tubes and chemicals needed for processing, it still relies on laboratory infrastructure (namely a centrifuge) for implementation. The FASTPlaque-Response™ tuberculosis diagnostic kit is used for detection and determination of rifampicin resistance of the tuberculosis bacteria. The preparation procedure is also complex and requires centrifugation.

The requirement of laboratory infrastructure and technical expertise poses a burden in any clinical setting and these requirements and associated costs make sample preparation impossible to implement in many resource-limited settings.

There is a need for quicker and more reliable bacterial diagnostics (products, kits, and methods) that can be feasibly implemented, and that go beyond the bacteriophage-host binding infecting event. This need includes bacterial diagnostics that are more specific, sensitive, accurate, and reproducible, as compared to conventional bacterial diagnostics. There is also a need to further exploit the intrinsic high specificity of bacteriophages to their corresponding host bacteria.

In addition, there is also a need for a sample preparation apparatus that is low in cost, easy to use, and does not require technical expertise or any additional laboratory infrastructure to perform.

### BRIEF SUMMARY OF THE INVENTION

It is therefore an object and advantage of the present invention to provide less costly, more efficient, more specific, faster, more accessible, and better adaptable processes and apparatuses for selective organism (e.g., bacterial) detection than provided by currently available technology.

It is also an object and advantage of the present invention to provide bacterial diagnostics (products, kits, devices, and methods) that further exploit the intrinsic high specificity of bacteriophages to their corresponding host bacteria, as compared with conventional bacterial diagnostics.

In accordance with the foregoing objects and advantages, an embodiment of the present invention provides recombinant bacteriophages, a method for constructing and producing such recombinant bacteriophages, and use of such recombinant bacteriophages for detecting target bacteria and/or for determining drug(s)/antibiotics to which the target bacteria is resistant.

In accordance with an embodiment of the present invention, products, kits, and methods that are capable of detecting specific types of bacteria, for example, by probing for the presence of specific nucleic acid sequences and/or genes within (that is characteristic of) a targeted viable bacterium rather than merely through the bacteriophage/host binding/infecting event, are provided. Generic examples of such products/methods may include those based on: bacterial culture; bacterial staining and microscopy; enzyme-Linked ImmunoSorbent Assay (ELISA); polymerase chain reaction (PCR); and other bacteriophage-based methods.

It is further object and advantage of the present invention to provide the capability to probe other specific nucleic acid sequences in order to detect for characteristics that, for example, give rise to a bacterium's drug resistance. Thus, the detection of a specific bacterial nucleic acid sequence can be made dependent upon the expression and detection of the reporter gene(s) used.

In accordance with an embodiment with the present invention, a method to detect specific nucleic acid sequences in a target (i.e. viable bacteria) in a sample comprises the following steps that may occur alone or in combination, as appropriate: (a) the bacterium in the sample is exposed to infection by genetically engineered bacteriophages, which have had their lytic cycle repressed or deleted. Reporter gene(s) are incorporated in the genome of the bacteriophage. This reporter gene(s) is placed downstream of a promoter and flanked by nucleic acid sequences homologous to a target nucleic acid sequence to be detected in the bacteria; (b) the infected bacteria express the reporter gene(s) only if the target nucleic acid sequence(s) or gene(s) is/are present in the bacteria, and homologous recombination with gene replacement occurs; and (c) the reporter gene(s) may then be detected directly or indirectly.

In accordance with an embodiment with the present invention, in vitro diagnostic kits and devices for detecting target bacterial organisms are provided.

In accordance with an embodiment with the present invention, sample preparation apparatuses for preparing samples for detection and analysis using bacteriophage-based techniques, that are low in cost, easy to use, and do not require technical expertise or any additional laboratory infrastructure to perform, are provided.

### BRIEF DESCRIPTION OF THE DRAWINGS

The present invention will be more fully understood and appreciated by reading the following Detailed Description in conjunction with the accompanying drawings, in which:

FIG. 1 is a graphical illustration of the construction of a recombinant bacteriophage, according to an embodiment of the present invention, where the recombinant bacteriophage can be used to detect the bacterial cells presence in a sample and its resistance to specific drugs.

FIG. 2 is a graphical illustration of the production of a recombinant bacteriophage(s), according to an embodiment of the present invention.

FIG. 3 is a graphical illustration showing the utilization of the bacteriophage(s) produced as shown in FIG. 2 (which may be present in a test kit) to test whether a sample contains target bacterium, and to test the bacterial drug sensitivity profile of the target bacterium, according to an embodiment of the present invention.

FIG. 4 is a graphical illustration of the use of a lateral flow device as a specific example of detecting target bacteria and/or drug resistant target bacteria in a biological sample, according to an embodiment of the present invention.

FIG. 5a is a graphical illustration showing a bacteriophage probing construct comprising a primary reporter gene (RG), which is placed downstream of a promoter (P) and flanked by nucleic acid sequences (5′ HT; 3′ HT) homologous to a specific target nucleic acid sequence (Target) to be detected within a target bacterium, according to an embodiment of the present invention.

FIG. 5b is a graphical illustration showing a gene replacement event from a double crossover event between the homologous nucleic acid sequences (5′ HT; 3′ HT) flanking the reporter gene (RG) in the bacteriophage as shown in FIG. 5a, and the specific target gene in the target bacteria genome, according to an embodiment of the present invention.

FIG. 6 is an illustration showing a bacterial in vitro diagnostic kit, and related diagnostic method, for detecting target bacteria in a sample through the use of genetically engineered bacteriophages, according to an embodiment of the present invention.

FIG. 7 is a graphical diagram illustrating a nasal swab sampling apparatus in conjunction with a lateral flow device, according to an embodiment of the present invention.

FIG. 8 is a side perspective view of a ‘lab within a syringe’ apparatus, according to an embodiment of the present invention.

FIG. 9 is a brief illustration of a sample preparation procedure for preparing samples for detection and analysis using bacteriophage-based techniques, and using the sample preparation apparatus as set forth in FIG. 8, according to an embodiment of the present invention.

FIG. 10 is a more detailed illustration of a sample preparation procedure for preparing samples for detection and analysis using bacteriophage-based techniques, and using the sample preparation apparatus as set forth in FIG. 8, according to an embodiment of the present invention.

FIG. 11 is a front perspective view of a lab within a syringe apparatus, according to an embodiment of the present invention.

FIG. 12 is a side perspective view of a lab within a tube apparatus, according to an embodiment of the present invention.

FIG. 13 is a side perspective view of the lab within a tube apparatus as shown in FIG. 12, comprising a syringe component and a main chamber device component, where the syringe component is inserted within the main chamber device component, according to an embodiment of the present invention.

### DETAILED DESCRIPTION OF THE EMBODIMENTS OF THE INVENTION

Embodiments of the invention will be more fully understood and appreciated by reading the following Detailed Description in conjunction with the accompanying drawings, wherein like reference numerals refer to like components.

The technology underlying the embodiments of the present invention herein is based on the intrinsic high specificity of bacteriophages (i.e. viruses that infect bacteria) to their corresponding host bacteria, as discussed supra.

In accordance with an embodiment of the present invention, the process of employing phages capable of being detected via a reporter molecule that is fused to the phage, benefits from a production strategy that produces a phage that lacks the expressed reporter molecule, but contains the reporter molecule DNA. After infection, however, the phage progeny incorporate the expressed reporter molecule. Small molecules (i.e. smaller than 40 KDa), such as small proteins, peptides, epitopes, and oligomers with desired characteristics that allow for their detection (e.g. high binding affinity, immunogenicity, chemical reactivity, conductivity, electrochemical activity, etc) may be used as reporter molecules, although larger molecules with the desired characteristics may also be used if desired.

Some of the advantages of an embodiment of the present invention when compared with the conventional bacterial detection methods and products include: (i) homologous recombination with gene replacement adds additional specificity and functionality to the detection system of an embodiment of the present invention by allowing the detection of specific sequences in the genomic DNA of the target bacterium; (ii) the ability to produce a recombinant bacteriophage without the presence of any of the reporter molecule(s) used in the detection system, but possessing in the bacteriophage DNA sequence(s) coding for one or more of the reporter molecule(s) controlled by different conditional promoter(s). The presence of the different conditional promoters each individually controlled adds more sensitivity to the present system. No reporter molecule will be present prior to the infection of the target bacterium by the recombinant bacteriophage, minimizing false positive results. In addition to the increased sensitivity of the present method, the use of reporter molecules controlled by different conditional promoters allows a rational control of the reporter molecules expression by the presence or absence of the promoter's inducers or repressors at different steps in the detection protocol. This rational control incorporates the ability to detect the susceptibility of the infected target bacterium to different conditions and/or treatments, such as the presence of drugs like antibiotics or bactericides and/or to physical treatments like temperature into the system.

Advantages of the invention are illustrated by the following Examples. However, the particular materials and amounts thereof recited in these examples, as well as other conditions and details, are to be interpreted to apply broadly in the art and should not be construed to unduly restrict or limit the invention in any way.

### Example 1

This Example describes the construction of a recombinant bacteriophage, in accordance with an embodiment of the present invention. The construction of a recombinant bacteriophage comprises the modification of the bacteriophage's genome. The bacteriophage's genome (e.g. Mycobacterium species bacteriophages: L5, D29, TM4, Bxb1, DS6A, Barnyard, Bxz1, Bxz2, Che8, Che9c, Che9d, Cjw1, Corndog, Omega, Che12, Bethlehem, and U2; Staphylococcus aureus bacteriophages: P1, P14, CDC 47, 42E, CDC 52, CDC 52A, CDC 79, CDC 53, and UC 18; Enterococcus faecalis bacteriophages: VD13, 42, phiEF24C, PlyV12, and phiFC1; Clostridium difficile bacteriophages: phiC2, phiCD119, PhiC5, PhiC6, PhiC8, C2, and CD630) is modified such that one or more bacteriophage's gene(s) is/are controlled by conditional promoter(s) (e.g. heat shock promoters, where 42° C. is the restrictive temperature and the permissive temperature can be, for example, room temperature to 37° C. Conditional promoters may also be associated with growth phase, stage of infection, and growth conditions, such as presence or absence of a nutrient. One example of a promoter repressed by the presence of a chemical agent (repressor) is the xylR P_{xylA} promoter system where PxylA is repressed by xylose in Staphylococcus aureus. In contrast, the Pcad promoter system is induced by the presence of cadmium in Staphylococcus) and can be fused to genes that code for Detectable Reporter Molecules, such as peptide, protein, DNA, and/or RNA. Furthermore, these molecules can be designed to generate a detectable electrical, chemical, or optical signal and/or to exhibit a specific affinity to other molecules that can be used to capture the reporter molecules. In addition, the reporter molecules can be naturally occurring molecules such as fluorescent proteins and antibodies in the case of proteins or ribozymes and copies of the host or phage DNA or RNA in the case of nucleotides. In addition, the reporter molecule can be synthetically designed such as small peptides or oligomers designed to generate specific electrical, chemical, or optical signals and/or designed to exhibit affinity to other molecules as well as fragments of antibodies. Oligomer-derived reporter molecules could be designed to confer specific affinity to other molecules simply based on affinity to complementary nucleotide sequences and/or through designing oligomers to generate aptamers with specific affinity to a variety of molecules and other materials). Exogenous DNA or RNA fragments coding for Detectable Reporter Molecule(s), is/are incorporated into a bacteriophage's DNA or RNA and are autonomously controlled by conditional promoter(s).

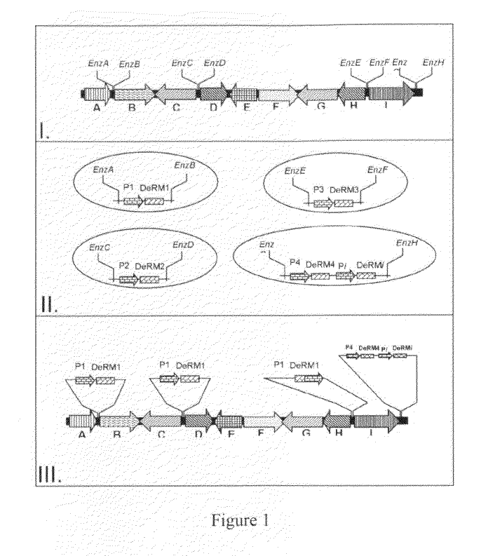

As seen in FIG. 1, a graphical illustration of the construction of a recombinant bacteriophage is shown in three sections (I, II, III), according to an embodiment of the present invention. In section (I), a diagram representing an example of a map of a bacteriophage genome is shown (e.g. Mycobacterium species bacteriophages: L5, D29, TM4, Bxb1, DS6A, Barnyard, Bxz1, Bxz2, Che8, Che9c, Che9d, Cjw1, Corndog, Omega, Che12, Bethlehem, and U2; Staphylococcus aureus bacteriophages: P1, P14, CDC 47, 42E, CDC 52, CDC 52A, CDC 79, CDC 53, and UC 18; Enterococcus faecalis bacteriophages: VD13, 42, phiEF24C, PlyV12, and phiFC1; Clostridium difficile bacteriophages: phiC2, phiCD119, PhiC5, PhiC6, PhiC8, C2, and CD630). The capital letters below the diagram, from A to I, illustrate the different Open Reading Frames that may be available in the bacteriophage genome, represented by the different arrows. Examples of different Restriction Sites in the genome are shown as an illustration, which are represented by the code Enz followed by a capital letter.

Section (II) Shows different possible constructions that could be used, each coding for one or more Detectable Reporter Molecule (DeRM) (e.g. MBP, GST, HP thioredoxin, V5 epitope, GB1, poly-Pro-Phe-Tyr, and 6×HisTag) downstream of Autonomous Promoters (P) with desired characteristics (e.g. constitutive, conditional, etc). Each construction may be flanked with sequences homologous with specific target genes in a bacterium of interest (e.g. Enterococcus faecalis vanA and vanC1, associated with Vancomycin resistance; Staphylococcus aureus mecA, associated with methicillin resistance; Mycobacterium tuberculosis katG, gyrA, gyrB, and inhA associated with Isoniazid resistance, and pstB, Rv1258c, Rv1410c, and other efflux pumps that may be associated with one or more of the following antibiotics, rifampicin, isoniazid, ethambutol, and streptomycin; Clostridium difficile gyrA, gyrB, and efflux pump genes which are associated with resistance to Fluoroquinolones on this microorganism). Such flanking sequences are incorporated in the bacteriophage constructions in the case where gene-specific detection in the bacterium is desired. At the extremities of each construction, different restriction sites are shown. The examples shown in section (II), as well as the protocols used to insert one or more of these constructions into the bacteriophage genome shown in (I) can be achieved through conventional molecular cloning techniques and protocols as described by Sambrook et al., 1989. J. Sambrook, E. F. Fritsch and T. Maniatis, Molecular Cloning. In: (2nd ed.), A Laboratory Manual, Cold Spring Harbor Laboratory Press, Cold Spring Harbor, N.Y. (1989), which is hereby incorporated by reference herein in its entirety. A diagram representing a final product, with the various Detectable Reporter Molecule (II) constructions integrated into the bacteriophage genome (I) is shown in (III).

### Example 2

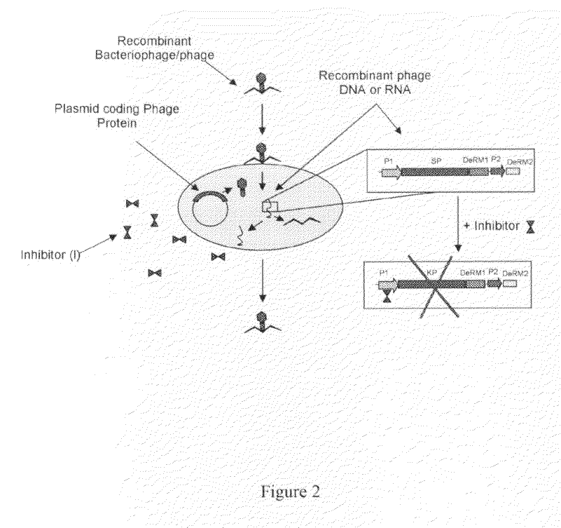

This Example describes the production of recombinant bacteriophages, in accordance with an embodiment of the present invention. Recombinant host bacteria are used to produce the bacteriophage. The recombinant host bacteria contain exogenous DNA coding for the wild type gene(s) with respect to the constructed recombinant bacteriophages described in Example 1, although without any of the described modifications and/or fusions. The recombinant bacteriophage is allowed to infect this recombinant host bacterium under conditions that neither allows the expression of the modified gene(s) in the constructed recombinant bacteriophage's genome described supra, nor the additional Detectable Reporter Molecules(s). Functional bacteriophages are then produced using the unmodified wild type proteins present in the recombinant phage genome and those in the recombinant host bacteria, without producing exogenous reporter molecule(s). This production of recombinant bacteriophages is further illustrated in FIG. 2, as discussed infra.

Turning to FIG. 2, a graphical illustration of the production of a recombinant bacteriophage is shown. The recombinant bacteriophage DNA contains a Detectable reporter molecule (DeRM1) which may or may not be fused to its structural protein (SP), and is controlled by an exogenous promoter (P1). Other Detectable Reporter molecule(s) (DeRM2, DeRMi) may also be present in the recombinant bacteriophage, and each one may be controlled by a different exogenous promoter (P2, Pi).

As shown in FIG. 2, the recombinant bacteriophage infects a recombinant bacterium, which contains a plasmid coding for the wild type (WT) structural protein(s) of the bacteriophage, in case any detectable reporter molecule is fused to the structural proteins of the recombinant bacteriophage. After infection, the lysogens are placed in a permissive condition (e.g., in the presence of a compound (e.g., Inhibitor (I)), which inhibits the expression of the structural protein(s) fused with the Detection Reporter Molecule (SP+DeRM1) and present in the recombinant bacteriophage DNA, and any other Detectable Reporter Molecules (DeRM2, DeRMi) fused or not to other(s) structural proteins.

With the expression of all the DeRM repressed, in case some DeRM were fused to any structural protein (SP) of the recombinant bacteriophage, the plasmid in the recombinant bacterium expresses the wild type structural protein without the fused DeRM. This structural protein expressed from the plasmid present into the bacteria and without any fusion is then assembled with the other bacteriophage protein(s) expressed from genes introduced by the bacteriophage. The result is a progeny bacteriophage absent of any Detectable Report Molecule(s).

The resultant recombinant bacteriophage that is produced can be used, for example, in a kit as pursuant to the methodology described in Example 3, infra. The resultant recombinant bacteriophage contains in its genome all of the necessary information to produce the different DeRM(s), but do not have any expressed detectable reporter molecule, and is not fused to any expressed structural protein.

### Example 3

This Example describes how the recombinant bacteriophages produced in Example 2 are used to detect the presence of target bacteria in a sample. The detection of target bacteria in a sample occurs through the expression of Detection Reporter Molecules, as described in Example 2.

The recombinant bacteriophages produced in Example 2 can be used to infect target bacterial cells that are present within a sample. The infected target bacterial cells (i.e. lysogens) are kept in a condition such that the exogenous Detection Reporter Molecules (described in Example 2) is expressed (e.g. presence of an inducer or absence of a repressor), thereby providing a means for identifying the target bacteria by detecting the expressed Detection Reporter Molecules.

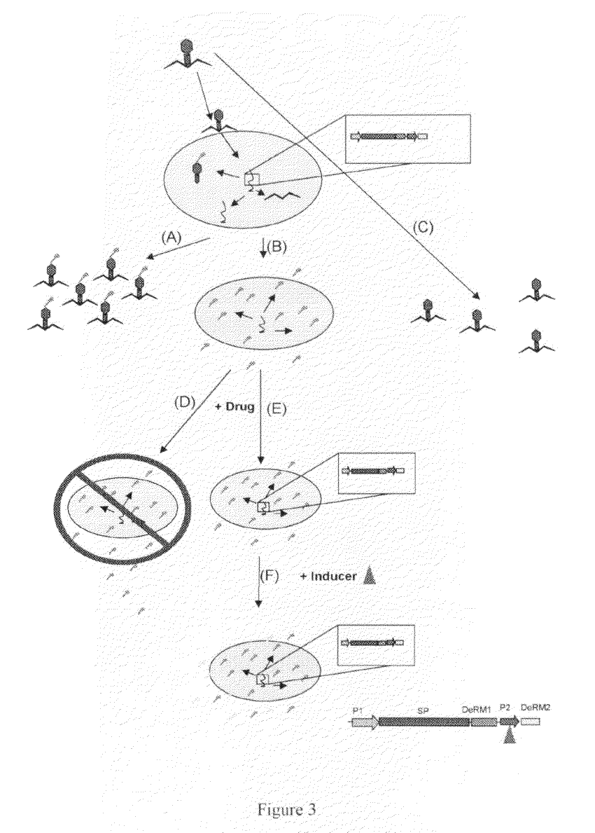

Turning to FIG. 3, the bacteriophage produced in Example 2 (which may be present in a test kit) is used to test whether a sample (e.g. blood, urine, sputum, etc) contains any target bacterium (e.g. Mycobacterium species, Staphylococcus species, Listeria species, Clostridium species, Enterococcus species, Streptococcus species, Helicobacter species, Rickettsia species, Haemophilus species, Xenorhabdus species, Acinetobacter species, Bordetella bronchisepta, Pseudomonas aeruginosa, Aeromonas species, Actinobacillus species, Pasteurella species, Vibrio species, Vibrio species, Legionella species, Bacillus species, Calothrix species, Methanococcus species, Stenotrophomonas species, Acinetobacter species, Chlamydia species, Neisseria species, Salmonella species, Shigella species, Campylobacter species, and Yersinia species). If the sample to be tested contains any target bacterium, the recombinant bacteriophage can undergo its lytic cycle (A), or its lysogenic cycle after infection (B), and in both cases the first reporter molecule (DeRM1) can be produced (this is because there is no inhibitor present that inhibits the expression of the protein fused with the Detection Reporter Molecule (SP+DeRM1), as described in Example 2). If the target bacteria is not present, however, the infection does not take place, and DeRM1 is not produced (C).

### Example 4

This Example describes a method of determining if specific target bacteria are resistant to a specific drug. In brief, the lysogens formed by the infection of the target bacteria with the recombinant bacteriophage (as described in Example 3), are exposed to a drug. After a pre-determined period of time, the expression of the Detectable reporter Molecule (as described in Example 2) is activated (e.g., by the addition of an inducer molecule or inactivation of a repressor), thus producing the Detectable Reporter Molecule. More than one Detectable Reporter Molecule can be present and controlled by different promoters, allowing the detection of bacteria's resistance to more than one drug. In this manner, if a drug does not affect the target bacteria, Detectable Reporter Molecules will be expressed. Detection of these Detectable Reporter Molecules thus allows for the determination of resistance by the target bacteria to specific drugs.

As illustrated in FIG. 3, the lysogenic cycle of the infected target bacteria can be exploited using a temperate phage, or recombinant phages that perform the lysogenic cycle in permissive and/or controlled conditions (B). In this case, the bacterial drug sensitivity profile can be tested (by exposing the bacteria to a drug).

After infection of the target bacteria that was present in the sample by the recombinant bacteriophage and production of DeRM1 after the recombinant bacteriophage entered its lysogenic cycle (as discussed in Example 3-(B)), the target bacteria are exposed to a drug to be tested. If the bacteria are sensitive to the drug (not resistant), they will be killed (become metabolically inactive) by the drug (D). If the bacteria are resistant to the drug being tested, they will survive (metabolically active).

After a pre-determined time following the drug treatment step (long enough for the drug to kill any sensitive bacteria), a molecule (Inducer) is added to the media (F). This inducer will act on the promoter controlling the second reporter molecule (DeRM2), inducing its production in the bacteria that survived the addition of the drug. Detection of DeRM2 indicates that the remaining viable target bacteria are resistant to the drug tested.

### Example 5

This example describes the use of a lateral flow device as a specific example of detecting target bacteria and/or drug resistant target bacteria in a biological sample. Lateral flow devices should be understood by those skilled in the art, and need not be described in great detail herein. Briefly, reporter molecules can be designed for use in a lateral flow device where the reporter molecules would exhibit affinity to dye particles for visualization on the lateral flow membrane, as well as affinity to other molecules immobilized on the lateral flow membrane, for localization on the lateral flow membrane. A solution containing such reporter molecules can then be applied to the lateral flow device where they may be conjugated to dye particles and then become localized by the immobilized molecules on the lateral flow membrane. A visual signal is generated by the localized accumulation of conjugated dye particles.

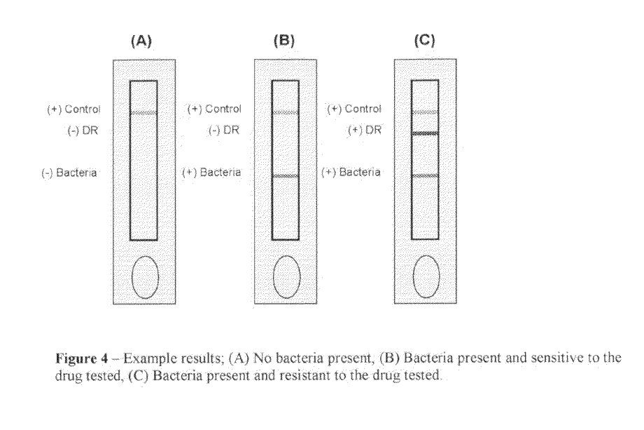

Turning to FIG. 4, strip (a) shows the presence of a bold line at the (+) control location, but without any lines in the (−) DR (“drug resistant bacteria”) or in the (−) target bacteria locations. Strip (a) indicates that the lateral flow test is working; however there are no target bacteria present, drug resistant or otherwise. Strip (b) shows the presence of a bold line at each of the (+) control and the (+) target bacteria locations, but without a lines in the (−) DR location. Strip (b) indicates that the lateral flow test is working, target bacteria are present, but that there are no drug resistant target bacteria present. Strip (c) shows the presence of a bold line at each of the (+) control, the (+) target bacteria, and the (+) DR locations. Strip (c) indicates that the lateral flow test is working, target bacteria are present, and drug resistant target bacteria are present.

An alternative embodiment of the invention will now be described with reference to the following Examples. This alternative embodiment provides a method to detect specific nucleic acid sequences within viable bacteria. This alternative embodiment also provides genetically modified bacteriophages.

In this detection method, target bacteria present in a sample (e.g., blood, sputum, urine, food, water, soil, etc.) are specifically infected by a genetically modified bacteriophage that has had its lytic cycle repressed or deleted, and that contains at least one reporter gene. The primary reporter gene is placed downstream of a promoter which may be conditional or constitutive and is flanked by nucleic acid sequences that are homologous to specific target nucleic acid sequences present within a bacterium forming a probing construct. Additional probing constructs may be included that may contain different reporter genes and can probe for different target nucleic acid sequences. In the presence of the target nucleic acid sequence(s), a gene replacement event takes place through a double crossover event between the homologous nucleic acid sequences of the bacteriophage and the target sequences present in the bacteria. This double crossover event results in the replacement of the target nucleic acid sequence present in the bacteria with the reporter gene included in the reporter construct. This will activate the reporter genes, which will be expressed by the bacteria making these expressed molecules available for detection and identification. Additionally, the probing construct may be regulated by a conditional promoter that may restrict expression of the reporter molecule in the presence of an inhibitor or in the absence of an inducer.

Pursuant to this method of an alternative embodiment of the present invention, a specific organism (e.g., specific target bacteria) can be detected by probing for nucleic acid sequences and/or genes that are characteristic of the organism; for example, when detecting the bacterium Escherichia coli, the signature nucleic acid sequence present in 16S rDNA fragments can be used as the flanking sequence.

Reporter genes introduced by an engineered bacteriophage will only be expressed if specific target nucleic acid sequence(s) within the specific host bacterium is/are present. In this manner a specific organism can be detected via probing for nucleic acid sequence(s) and/or gene(s) that is characteristic and specific to the organism, and not simply through the bacteriophage-host infection event. Since many bacteriophages may be specific for several different types of bacteria within a single genus, the use of this method is highly desirable when one specific type of bacteria within the genus needs to be identified. In other words, this bacteriophage detection method of an alternative embodiment of the present invention comprises another layer of increased specificity with respect to the identification of specific target bacteria within a sample.

Furthermore, pursuant to this method of an alternative embodiment of the present invention, other specific nucleic acid sequence(s) and/or gene(s) may be probed in order to detect genotypes (bacterial characteristics) that would indicate or give rise to a bacterium's drug resistance, or the ability to produce enzymes or other functional protein that would produce a phenotype of interest (such as the ability to grow on specific conditions, metabolize different substrates, or produce different metabolic products and/or proteins).

For example, the nucleic acid sequence of a Multidrug Efflux Pump can be used as the flanking sequence when probing for multidrug resistance in Pseudomonas aeruginosa. Thus, in order to diagnose an organism's drug resistance, the disclosed technique does not require the addition of the drug for selective purposes. In another example the nucleic acid sequence of a specific enzyme that allows the use of a specific substrate by the cell can be used as a flanking sequence. In this case the gene replacement event could act to simply delete this phenotype and analysis of the inability of the cells to metabolize the specific substrate could allow the determination of the presence of a targeted cell without the need for generating an exogenous reporting signal. In some cases, the target cells capability of producing an enzyme may result in the formation of toxic compounds, thus the detection of the genes in a target cell that code for such enzyme would result in a means of detecting a target cell's capability of producing a specific toxin. In another example, another phenotype of interest is the nucleic acid sequence that directly codes for the production of a specific toxin (e.g. enterotoxin A, B, or C of Staphylococcus aureus). In this case, toxin-coding sequences would constitute the flanking sequence when probing for the ability of bacteria species to produce specific toxins.

The genetically modified bacteriophage may contain genes that will increase the host's metabolism, for example, by the expression of proteins which will increase the bacteria's uptake of nutrients from the media. Further, the genetically modified bacteriophages may contain genes that will improve the expression of the reporter genes, for example, by the repression of genes involved in the secondary metabolism in the host cell, or by the expression of proteins directly involved in the protein synthesis machinery in the host cell. These “helper” genes will be activated and expressed as soon as the bacteriophage genomic material naturally establishes into the host bacteria's genome. The genetically modified bacteriophage may also contain genes that will increase the frequency of homologous recombination in the host bacteria's genome by the expression of RecA protein, for example. The genetically modified bacteriophage may contain a lytic cycle on its genome, but it can be conditionally controlled and/or repressed through the use of conditional promoters. Nucleic acid molecules that are introduced by the phage may be controlled by a variety of promoters that can be host specific and/or derived from the phage or other sources. For example, appropriate promoters may be chosen from a source that will confer over-expression of the phage-introduced nucleic acids.

An embodiment of the present invention provides a plurality of genetically engineered bacteriophages, each of which may specifically infect a different target bacterium, from different genera, species, or subspecies. A plurality of genetically modified bacteriophages can have different reporter genes that may be used together, and the presence of one or more target bacteria and/or one or more type of nucleic acid sequence and/or genes could be identified.

In accordance with an embodiment of the present invention, the reporter gene may be expressed by producing a fluorescent protein, for example, a green fluorescent protein, which remains inside of the host and can be detected through its optical excitation and resulting optical emission visually or via a light detection apparatus. Additionally, the reporter gene may be expressed and secreted by the host bacteria, which can then react with a component in the medium or within the host cell, or which catalyzes a reaction in the medium to give a detectable signal. The reporter gene may be expressed and secreted by the host bacteria and can then be sensed by a device or a device surface in order to be detected by electrical means through the device. The reporter genes used on the same bacteriophage may be a plurality of the reporter genes referred to above, and the product of the reporter genes used on the same bacteriophage could be differentiated (e.g., different colors of fluorescent protein).

### Example 6

This Example describes a method to detect specific nucleic acid sequences in a target (i.e. viable bacteria) by the use of genetically modified bacteriophages that have had their lytic cycle repressed or deleted, and that contain at least one reporter gene present in its DNA sequence (as described supra).

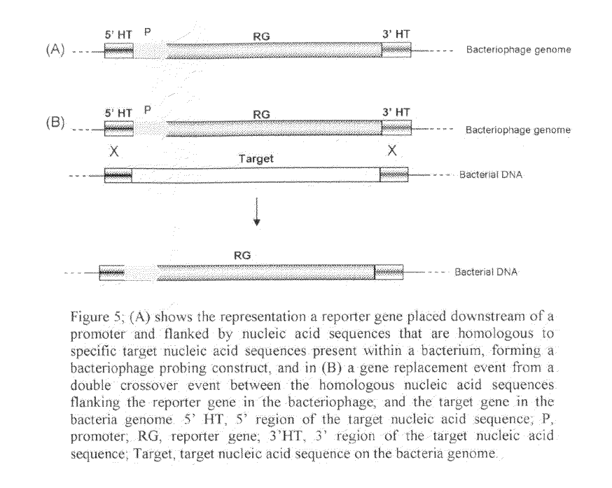

Turning to FIG. 5a, a graphical illustration of a bacteriophage probing construct comprising a primary reporter gene (RG), which is placed downstream of a promoter (P) and flanked by nucleic acid sequences (5′ HT; 3′ HT) homologous to a specific target nucleic acid sequence (Target) to be detected within a target bacterium, is shown. Additional reporter genes may be placed downstream of different promoters and flanking other specific nucleic acid sequence(s).

Turning to FIG. 5b, a graphical illustration of a gene replacement event from a double crossover event between the homologous nucleic acid sequences flanking the reporter gene in the bacteriophage, and the specific target gene in the target bacteria genome, is shown. In the presence of the target nucleic acid sequence(s) (Target), the gene replacement event arises from a double crossover event between the homologous nucleic acid sequences (5′ HT; 3′ HT) of the bacteriophage and the target sequence (Target) in the bacterium. This double crossover event results in the complete removal of the target nucleic acid sequence (Target) present in the bacteria, and the replacement of the target nucleic acid sequence (Target) present in the bacteria by the reporter gene (RG). This will in turn activate the reporter genes (RG) where they are expressed, generating reporter molecules that are available for detection and identification.

Accordingly, a specific organism (e.g., a specific bacteria) may be detected by probing for specific nucleic acid sequence(s) and/or gene(s) that is characteristic to the organism, and not simply through the bacteriophage-host infection event. Similarly, other specific nucleic acid sequence(s) and/or gene(s) may be probed in order to detect for characteristics that, for example, give rise to a bacterium's drug resistance.

In accordance with a further embodiment of the present invention, an additional application of the engineered (generically modified) bacteriophages relates to a detection apparatus or device. According to this embodiment, engineered bacteriophages may be located and immobilized (e.g., on a micron scale) on a surface (or substrate) in a manner that can be multiplexed resulting in localization of the reporter signals for the development of a multi-target bacterial sensing and analysis device. This embodiment provides a device with high specificity, lower limits of detection, and shorter turnaround times, all of which have benefits relating to potentially higher sensitivity and superior efficacy over conventional and/or existing technologies.

The genetically engineered bacteriophages may be held to a substrate/matrix, and may be localized within areas of any dimension that may be genetically modified to hold the target bacteria even after its infection, functioning analogously to an antibody. The substrate/matrix may include an antibody and/or aptamer, which specifically recognize, bind and hold to the target bacteria and/or any of its surface components. The antibody may be a polyclonal antibody or a monoclonal antibody and/or fragments thereof.

Different genetically modified bacteriophages could be held in a different and predetermined localized area of any dimension on a solid substrate. Each of the different bacteriophages may also be modified to hold the target bacteria on the substrate even after its infection, functioning analogously as an antibody.

Development of the genetically engineered bacteriophage, as discussed supra, can be accomplished via conventional genetic engineering, microbiological, and phage display techniques, tools, and reagents. Genetic engineering methods can be used to design the bacteriophage genome, microbiological methods can be used to produce as well as characterize the activity of the engineered phage and also the employed antibodies, and phage display methods can be used to control and optimize phage-host interactions as well as phage-surface interactions for their immobilization.

Development of immobilized phages can be facilitated by the design of the genetically engineered bacteriophage and accomplished via standard surface chemistry and microfabrication techniques—For example, through depositing drops of engineered bacteriophages suspended in a liquid medium onto a surface modified to contain primary amine-reactive N-hydroxysuccinimide (NHS) esters that would react with primary amines available on the engineered bacteriophages forming a covalent amide bond between the phage and the substrate.

Controlled localization in microscopic dimensions may be achieved through using tools such as the commercially available BioForce Nanosciences NanoArrayer System. This could also be accomplished through microfabrication techniques similar to those outlined in: Bhatnagar, P; Strickland, A D; Kim, I, et al. Integrated reactive ion etching to pattern cross-linked hydrophilic polymer structures for protein immobilization Applied Physics Letters, 90(14):144107, 2007, which is hereby incorporated by reference herein in its entirety.

Characterization of a device for detecting the presence of a specific nucleic acid sequence in a viable bacterial organism employing a fluorescent reporter protein as described supra, can be achieved using fluorescent microscopes.

An exemplary detection device, as described in the following Example, may be implemented, for example, as a tuberculosis in vitro diagnostic tool. The design benefits from the advantages of the disclosed technology (i.e., low limit of detection, short turnaround time, and high specificity) and is, in addition, lower in cost and simple to use.

### Example 7

This Example describes an embodiment of a device and diagnosis method of the present invention for detecting the presence of a specific nucleic acid sequence in a viable bacterial organism (pursuant to the method as described on Example 6).

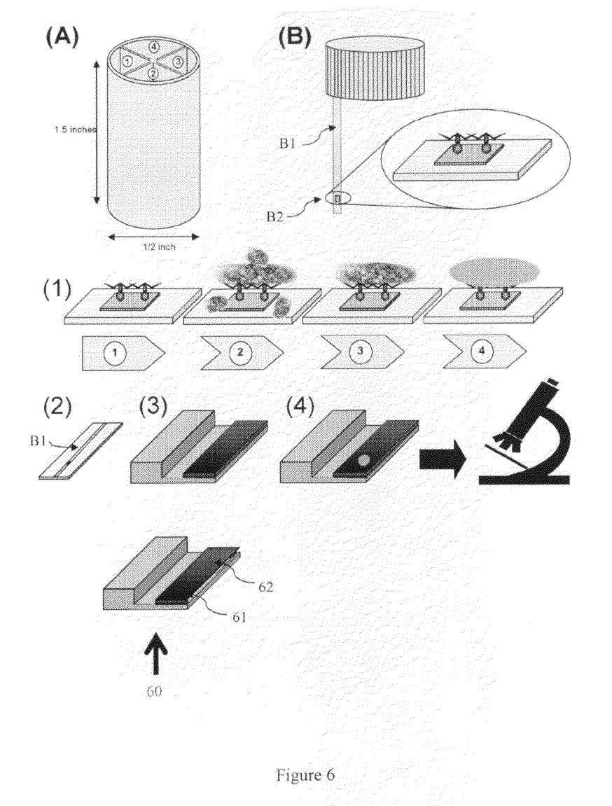

Turning to FIG. 6, a low cost, simple, bacterial in vitro diagnostic kit for detecting target bacteria in a sample through the use of genetically engineered bacteriophages is shown. The bacterial in vitro diagnostic kit can be suitable, for example, for implementation in developing countries. This diagnosis method associated with the bacterial in vitro diagnostic kit should not require any additional expertise or equipment other than conventional microscope slides and a conventional microscope.

FIG. 6 depicts a TB diagnostic kit. (A) Shows a container with a plurality of compartments. In particular, (A) shows a disposable tube with 4 compartments. (B) Shows the cap of this container and has a strip B1 attached to it with immobilized bacteriophages in a localized area B2 of micrometer dimensions. This micro patterning of bacteriophages could be achieved through depositing drops of engineered bacteriophages suspended in a liquid medium onto a surface modified to contain amine-reactive N-hydroxysuccinimide (NHS) esters that would react with amines available on the engineered bacteriophages, as discussed supra. Controlled, microscopic dimensions could be achieved through using tools such as the commercially available BioForce Nanosciences NanoArrayer System, also as discussed supra.

The bacterial in vitro diagnosis is performed as shown in FIG. 6 as follows: (1) the kit arrives with the strip B1 in compartment 1, dry. The sample from a patient and the strip B1 are inserted into compartment 2 that could contain a solution optimized for promoting phage-bacteria interaction. The strip B1 with bound bacteria is placed into compartment 3 which could contain a wash solution that may be composed of a mixture of salts and/or surfactants. The B1 with bound bacteria is then placed into compartment 4 which could contain a solution optimized for phage transfection and bacterial expression of a fluorescent probe molecule; (2) The strip B1 is removed from the cap and placed on a conventional microscope slide; (3) The slide with the strip B1 is inserted into the detection device 60 that includes light emitting diodes (LEDs) 61 that emit at wavelengths suitable for excitation of the fluorescent molecules as well as an optical filter 62 for detection of light emission from the probe. For example, this device could be constructed from plastic and include an LED emitting at around 488 nm and a filter that only allows the transmission of light at a wavelength around 509 nm, suitable for excitation and detection of the green fluorescent protein (GFP) molecule; and (4) The detection device is turned on, exciting the fluorescent probe and it is placed underneath a conventional (not fluorescent) microscope for detection of the fluorescent signal emanating from bacteria attached onto the micro patterned strip B1.

In accordance with an alternative embodiment of the diagnostic device, the main sensing platform (similar to the strip B1 described in FIG. 6) may consist of immobilized bacteriophages on a substrate where the bacteriophages are immobilized: (a) as a single or several type(s) of phage in a single localized area on the substrate; or (b) as a single or several type(s) of phage in several localized areas that can be in a specific pattern on the substrate where the different types of phage are in the same localized area or in separate localized areas. As discussed supra, this localized immobilization could be achieved through depositing drops of engineered bacteriophages suspended in a liquid medium onto a surface modified to contain amine-reactive N-hydroxysuccinimide (NHS) esters that would react with amines available on the engineered bacteriophages. Controlled localization in microscopic dimensions could be achieved through using tools such as the commercially available BioForce Nanosciences NanoArrayer System. This design allows for the detection of one or more organisms simultaneously that could be analyzed by recognizing a specific pattern through light emitting probe molecules derived from the phage-host interaction, or that could be recognized by different colors through the light emitting probe molecules localized in the same or separate areas.

In accordance with an embodiment with the present invention, other exemplary embodiments of the sensing platform are provided. For example, the sensing platform can be designed to be used with a sample of interest through a modified preparatory device (similar to the illustrative container described in FIG. 6) that will allow for any bacteria contained in the sample of interest to interact with the phages on the sensing platform. A preparatory device can be designed specifically for processing sputum samples for tuberculosis detection, and can include a sodium hydroxide solution in order to inactivate components in the sputum which may inhibit phage infection of the tuberculosis bacteria. This embodiment can further comprise a detector device that additionally contains magnifying optics designed to be analyzed directly and visually without the use of a separate microscope; this embodiment can also be designed to be used with external conventional detection equipment such as a fluorometer or fluorescence microscope for visual or automated analysis. The sensing platform of this embodiment can be designed to be inserted into a separate custom designed handheld detector device that allows for automated analysis. The sensing platform can also be designed to be used with a separate custom designed automated preparatory and detection device that automatically allows for any bacteria contained in a sample of interest (that is manually inserted into the preparatory and detection device or that is automatically acquired and inserted by the preparatory and detection device) to interact with the phages on the sensing platform, and then subsequently allow for analyses of the sensing platform.

In accordance with an embodiment with the present invention, sample preparation apparatuses for preparing samples for detection and analysis using bacteriophage-based techniques, that are low in cost, easy to use, and do not require technical expertise or any additional laboratory infrastructure to perform, are provided.

An exemplary autonomous bacterial sample preparation device may incorporate one or more compartments that may be separated by filters with pore sizes ranging from millimeters to sub-micrometers, one or more chemical agents that may be physically separated into the different compartments, and mechanisms for transferring fluid between the different compartments. The exemplary bacterial sample preparation device may further include an outlet port that may be sealed and which may contain a filter membrane of sub-micrometer pore sizes that retains bacteria within the device but allows liquid and small molecules to exit the device.

This exemplary bacterial sample preparation device will have the capability to:

(a) Homogenize said sample

(b) Neutralize and/or separate contaminants within said sample

(c) Capture bacteria within said sample

(d) Concentrate said bacteria

This exemplary bacterial sample preparation device can be designed to process, without limitation thereto, environmental samples, plant samples, veterinary samples, food samples, livestock samples, and medical samples. Such samples may further include soil samples, water samples, vegetable samples, meat samples, blood samples, urine samples, tissue biopsy samples, mucus samples, fecal samples, and sputum samples.

This sample preparation device will include the capability to stain target bacteria within the device with a dye incorporated within the sample preparation device. In another aspect, this exemplary bacterial sample preparation device can be designed to incorporate a solution of recombinant bacteriophages specific for target bacteria, such as the recombinant bacteriophages described herein, and will designed to allow the infection of the bacteria by the bacteriophages within the device and to allow production of reporter molecules in the case where the recombinant bacteriophages described herein are used.

This exemplary device can additionally allow for the lysis, extraction, and separation of macromolecules that may include proteins, peptides, oligomers, DNA, RNA, and lipids from within bacteria processed with the device.

This exemplary device may also integrate a lateral flow device designed to detect reporter molecules generated from the infection of target bacteria within the device by recombinant bacteriophages described herein and/or other macromolecules that may be released from lysis of the target bacteria. This lateral flow device could be integrated into the sample preparation device such that the molecules released from the target bacteria are applied to the lateral flow device upon breakage of the seal on the outlet of the sample preparation device.

Three additional exemplary embodiments of the apparatus for preparing samples for detection and analysis using bacteriophage-based techniques are described herein. These embodiments allow for chemical processing and concentration of bacteria in a sputum sample.

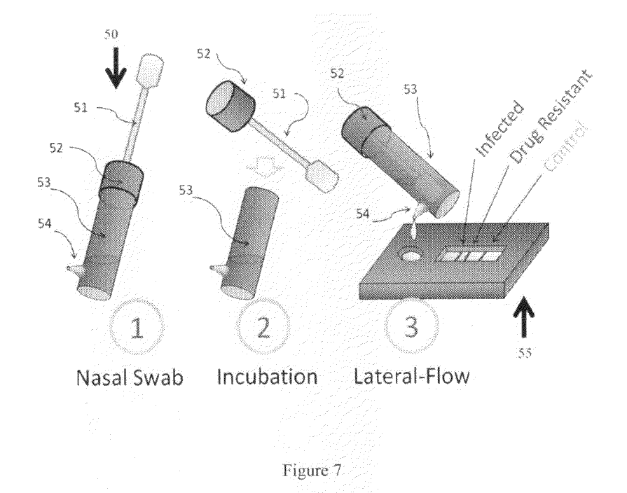

Turning to FIG. 7, a nasal swab sampling apparatus 50 in conjunction with a lateral flow device 55 is shown. The nasal swab apparatus 50 consists of a nasal swab 51 that is attached to the cap 52 of a tube 53. The tube may contain a solution of bacteriophages as described herein. The device also has an outlet 54 that may contain a <0.2 μm filter membrane. FIG. 7 further demonstrates the use of such a device. In step 1, the nasal swab 51 is used to swab the nose of a patient being tested for MRSA, for example. In step 2, the cap 52 is removed from the tube 53 and replaced back onto the tube 53 but with the nasal swab 51 placed into the tube 53. The bacteriophages inside the tube 53 would be allowed to infect the target bacteria that may be present on the nasal swab 51. In step 3, after reporter molecules are produced inside the tube 53 due to the bacteriophage infecting the target bacteria, the tube 53 could be squeezed in order to break a seal connecting the inside of the tube 53 to the outside of the tube 53 via the outlet 54. The solution inside the tube 53 containing the reporter molecules could be squeezed out of the tube 53 via the outlet 54 and into a lateral flow device designed for visual detection of the generated probe molecules.

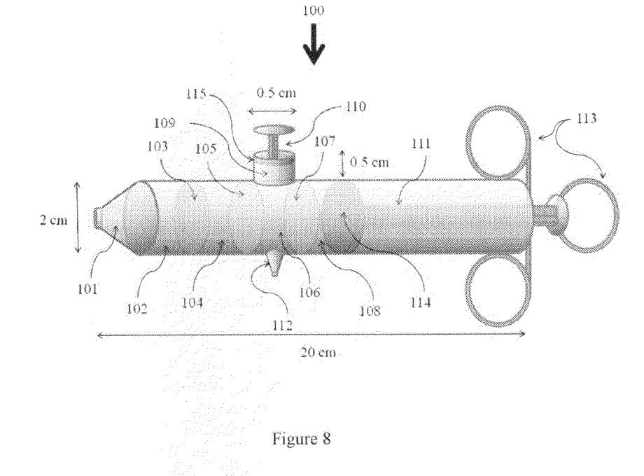

Turning to FIG. 8, a side perspective view of a ‘lab within a syringe’ apparatus 100 is shown. The apparatus 100 can comprise, without limitation, an inlet 101 (may determine max sample volume), a chamber I 102 (1% w/v NaOH powder), a macroscopic mesh 103 (help homogenize sample), a chamber II 104, a >0.45 μm uni-directional pore membrane 105 (allow bacteria to pass), a chamber III 106, a <0.22 μm pore membrane 107 (retain bacteria), a chamber IV 108 (gelling and neutralizing powder), a side chamber 109 (contains phage in media), a side chamber plunger shaft 110, a main plunger shaft 111, an outlet 112 (contains 0.22 μm pore membrane), finger holes 113 (for drawing out Main Plunger), a main plunger rubber 114, and a side chamber plunger rubber 115.

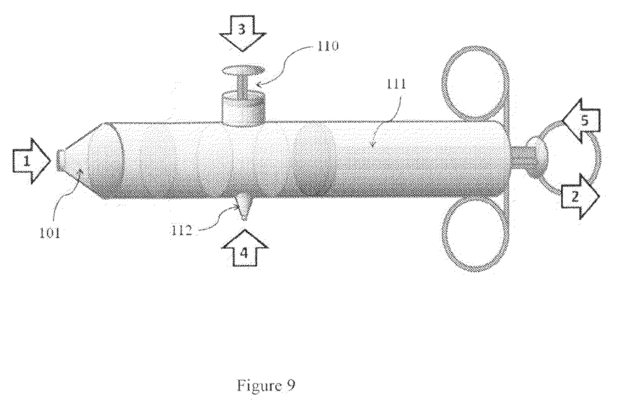

Turning to FIG. 9, a brief illustration of a sample preparation procedure for preparing samples for detection and analysis using bacteriophage-based techniques, and using the sample preparation apparatus as set forth in FIG. 8, is shown. The following steps of the procedure illustrated by numbers 1-5, without limitation, are as follows: (1) insert sample into the inlet 101, (2) pull the main plunger 111 out, (3) push the side plunger 110 in, (4) push the outlet 112 in (which connects chamber III 106 to outlet 112), and (5) push the main plunger 111 in.

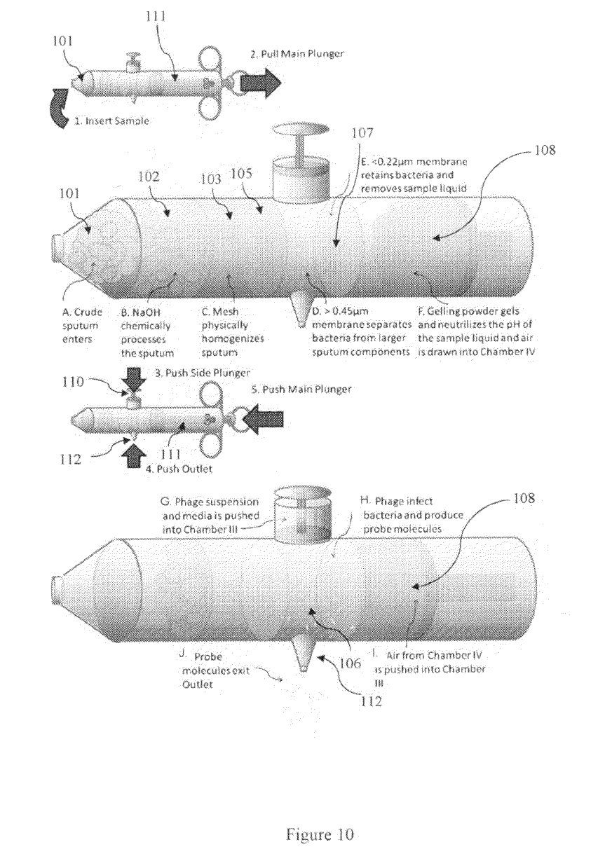

Turning to FIG. 10, a more detailed illustration of a sample preparation procedure for preparing samples for detection and analysis using bacteriophage-based techniques, and using the sample preparation apparatus as set forth in FIG. 8, is shown. The following steps of the procedure, without limitation, can be as follows: (1) insert a sputum sample into the inlet 101, (2) pull the main plunger 111 out, (A) crude sputum enters the inlet 101, (B) NaOH chemically processes the sputum in chamber I 102, (C) a macroscopic mesh 103 physically homogenizes sputum, (D) a >0.45 μm uni-directional pore membrane 105 separates bacteria from larger sputum components and bacteria passes into chamber III 106, (E) a <0.22 μm pore membrane 107 retains bacteria in chamber III 106 and removes sample liquid, and (F) gelling powder gels and neutralizes the pH of the sample liquid in chamber IV 108 and air is drawn into Chamber IV 108, (3) push the side plunger 110 in, (G) phage suspension and media is pushed into chamber III 106, (H) phage infect bacteria and produce probe molecules, (4) push the outlet 112 in to connect chamber III 106 to outlet 112, (5) push the main plunger 111 in, (I) air from chamber IV 108 is pushed into chamber III 106, and (J) probe molecules exit outlet 112.

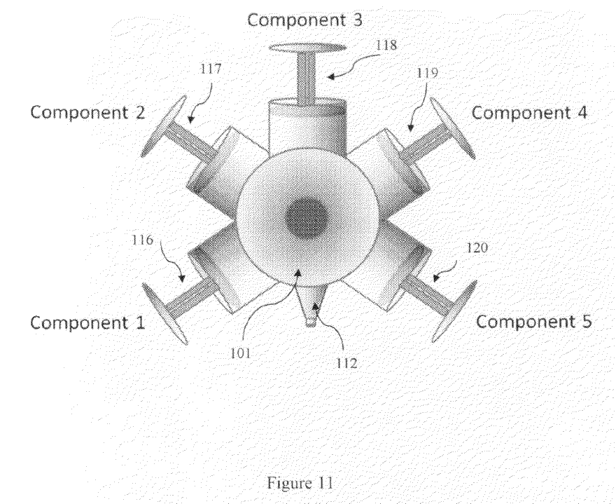

Turning to FIG. 11, a front perspective view of the lab within a syringe apparatus 100 is shown with additional side chambers. The apparatus can comprise at least one additional side chamber (e.g., 116-120 as shown in FIG. 11). Additional final treatments can be administered to the processed bacteria by adding these additional side chambers. For example, drug susceptibility can be assessed by adding drugs and other required components for determining the drug resistance profile. Several drugs can be incorporated to determine resistance of the bacteria to multiple drugs. In addition, a final step can incorporate a component to kill the bacteria and gel the solution that is left in chamber III 106. This final step would decontaminate the kit and allow one to dispose of it without hazard.

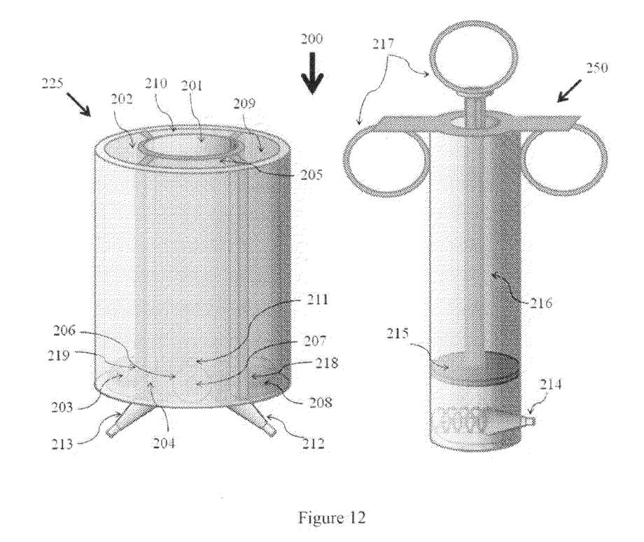

Turning to FIG. 12, a side perspective view of a lab within a tube apparatus 200 is shown. The apparatus 200 can comprise, without limitation, (1) a main chamber device 225 comprising a center chamber 201, chamber I 202 (contains 1% w/v NaOH powder), chamber I macroscopic mesh 203, chamber I/chamber II connection 204, chamber II 205, chamber II >0.45 um pore membrane 206, chamber II/center chamber connection 207, chamber III <0.2 um pore membrane 208, chamber III 209, chamber IV 210 (contains phage in media), chamber IV/center chamber connection 211, center chamber outlet 212 (contains <0.22 um pore membrane), center chamber inlet 213, and (2) a syringe 250 comprising a center chamber/side chamber connector 214, plunger rubber 215, a plunger shaft 216, and finger holes 217 (for drawing out Main Plunger).

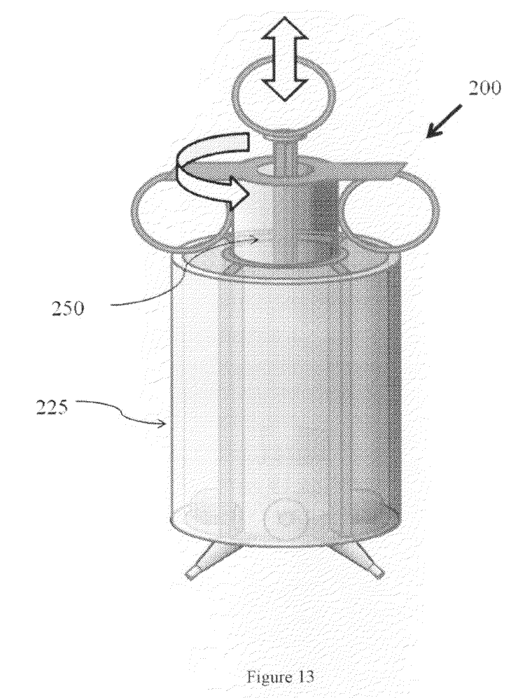

Turning to FIG. 13, a side perspective view of the lab within a tube apparatus 200 with the syringe 250 inserted within the main chamber device 225, and twisting motions being applied to the syringe 250, is shown.

A sample preparation procedure for preparing samples for detection and analysis using bacteriophage-based techniques, and using the sample preparation apparatus as set forth in FIG. 12, is shown. The following steps of the procedure, without limitation, can be as follows: the apparatus 200 starts with the syringe 250 in a position that is not connecting any chambers inserted into the center chamber 201; twist the syringe such that the center chamber/side chamber connector 214 connect the center chamber 201 with chamber I 202 through the chamber I/center chamber connection 219; push the plunger 216 down—the sample is forced through the chamber I macroscopic mesh 203 and into chamber I 202 where it mixes with the NaOH powder producing a 1% w/vNaOH sample solution (the sample is homogenized and chemically processed); twist the syringe 250 such that the center chamber/side chamber connector 214 connects the center chamber 201 with chamber II 205 through the chamber II/center chamber connection 207; pull the plunger 216 up—the sample is transferred from chamber I 202 into chamber II 205 through the chamber I/chamber II connection 204 and then back into the center chamber 201 through the chamber II >0.45 um pore membrane 206 (only the bacteria and small sample components pass into the center chamber); twist the syringe 250 such that the center chamber/side chamber connector 214 connect the center chamber 201 with chamber III 208 through the chamber III/center chamber connection 218; push the plunger 216 down—the sample is transferred from the center chamber 201 into chamber III 208 through the chamber III <0.22 um pore membrane 208 (the bacteria is retained in the center chamber 201); twist the syringe 250 such that the center chamber/side chamber connector 214 connect the center chamber 201 with chamber IV 210 through the chamber IV/center chamber connection 211; pull the plunger 216 up—the phage and media is transferred from chamber IV 210 into the center chamber 201 through the chamber IV/center chamber connection 211 (the phage infects the bacteria and produces reporter molecules); twist the syringe 250 such that the there is no connection made to any chamber; push the plunger 216 down—the probe molecules exit through the center chamber outlet 212. Additional processes can be integrated into this apparatus 200 by adding additional chambers, similar to those described for the lab within a syringe apparatus 100.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the spirit and scope of the appended claims.

What is claimed is:

1-15. (canceled)

16. A recombinant bacteriophage that is specific to at least one target bacterium, comprising:
an exogenous nucleic acid sequence encoding a first reporter molecule that is flanked by a first flanking nucleic acid sequence comprising a nucleic acid sequence homologous with a first target nucleic acid sequence within the at least one target bacterium.

17. The recombinant bacteriophage of claim 16, wherein said exogenous nucleic acid sequence encoding said first reporter molecule is operatively linked downstream to a promoter.

18. The recombinant bacteriophage of claim 17, further comprising a second flanking nucleic acid sequence comprising a nucleic acid sequence homologous with a second target nucleic acid sequence within the at least one target bacterium.

19. (canceled)

20. The recombinant bacteriophage of claim 18, wherein at least said first flanking nucleic acid sequence comprises a nucleic acid sequence homologous to a nucleic acid sequence of a target gene within said bacterium encoding at least one of a target phenotype and a strain specific signature sequence, including at least one of a drug resistance and a toxin production.

21-26. (canceled)

27. The recombinant bacteriophage of claim 16, wherein said first reporter molecule is adapted to generate a detectable signal, wherein said detectable signal is selected from a group including at least one of an electrical signal, a chemical signal, an optical signal, and a detectable affinity to a second molecule.

28. The recombinant bacteriophage of claim 1, wherein said first reporter molecule is a peptide that exhibits a specific affinity to at least one of another molecule or material, a protein such as antibodies or a fragment thereof, an enzyme capable of generating a detectable signal, and a fluorescent protein.

29. (canceled)

30. The recombinant bacteriophage of claim 16, wherein said reporter molecule is fused to an endogenous protein of a progeny of said recombinant bacteriophage.

31. The recombinant bacteriophage of claim 16, wherein said reporter molecule is fused to an endogenous protein of said at least one target bacterium.

32. The recombinant bacteriophage of claim 16, wherein said first reporter molecule includes at least one of DNA and RNA such as an oligomer of a specific sequence, a ribozyme, and an aptamer.

33. The recombinant bacteriophage of claim 16, wherein said recombinant bacteriophage exhibits at least one of a natural lysogenic cycle and a natural lytic cycle.

34. The recombinant bacteriophage of claim 16, wherein said recombinant bacteriophage exhibits at least one of a conditional lysogenic cycle and a conditional lytic cycle.

35. The recombinant bacteriophage of claim 30, wherein the expression of said fused reporter molecule is controlled by a conditional promoter.

36. The recombinant bacteriophage of claim 16, wherein the bacteriophage is derived from the group which infects at least one of Mycobacterium species, Staphylococcus species, Listeria species, Clostridium species, Enterococcus species, Streptococcus species, Helicobacter species, Rickettsia species, Haemophilus species, Xenorhabdus species, Acinetobacter species, Bordetella bronchisepta, Pseudomonas aeruginosa, Aeromonas species, Actinobacillus species, Pasteurella species, Vibrio species, Vibrio species, Legionella species, Bacillus species, Calothrix species, Methanococcus species, Stenotrophomonas species, Acinetobacter species, Chlamydia species, Neisseria species, Salmonella species, Shigella species, Campylobacter species, and Yersinia species.

37-62. (canceled)

63. A method of detecting the presence of target bacteria in a sample, comprising the steps of:
contacting said sample with a recombinant bacteriophage specific to said target bacteria comprising an exogenous nucleic acid sequence encoding a first reporter molecule that is flanked by a first flanking nucleic acid sequence comprising a nucleic acid sequence homologous with a first target nucleic acid sequence within the at least one target bacterium; and
assaying said sample for expression of said first reporter molecule, wherein expression of said first reporter molecule is indicative of possible presence of said target bacteria within the sample.

64. The method of claim 63, wherein said first flanking nucleic acid sequence is adapted to perform a crossover event after introduction of said first flanking nucleic acid sequence into said target bacterium wherein said first flanking nucleic acid sequence replaces said first target nucleic acid sequence within the at least one target bacterium.

65. The method of claim 64, wherein said recombinant bacteriophage further comprises a second flanking nucleic acid sequence comprising a nucleic acid sequence homologous with a second target nucleic acid sequence within the at least one target bacterium.

66. The method of claim 65, wherein said second flanking nucleic acid sequence is adapted to perform a crossover event after introduction of said second flanking nucleic acid sequence into said target bacterium wherein said second flanking nucleic acid sequence replaces said second target nucleic acid sequence within the at least one target bacterium.

67. The method of claim 66, wherein said exogenous nucleic acid sequence encoding a first reporter molecule is adapted to replace a third nucleic acid sequence of said target bacteria between said first and said second target nucleic acid sequences, wherein said replacement of said third nucleic acid sequence is triggers expression said first reporter molecule.

68. The method of claim 64, further comprising the step of inactivating a lytic cycle of said recombinant bacteriophage.

69-71. (canceled)

72. The method of claim 63, wherein said target bacteria comprises a bacteria species selected from the group consisting of Mycobacterium species, Staphylococcus species, Listeria species, Clostridium species, Enterococcus species, Streptococcus species, Helicobacter species, Rickettsia species, Haemophilus species, Xenorhabdus species, Acinetobacter species, Bordetella bronchisepta, Pseudomonas aeruginosa, Aeromonas species, Actinobacillus species, Pasteurella species, Vibrio species, Vibrio species, Legionella species, Bacillus species, Calothrix species, Methanococcus species, Stenotrophomonas species, Acinetobacter species, Chlamydia species, Neisseria species, Salmonella species, Shigella species, Campylobacter species, and Yersinia species.

73-75. (canceled)

76. The method of claim 63, wherein said sample is selected from the group consisting of environmental samples, plant samples, veterinary samples, food samples, livestock samples, and medical samples.

77. The method of claim 76, wherein said sample is selected from the group consisting of soil samples, water samples, vegetable samples, meat samples, blood samples, urine samples, tissue biopsy samples, mucus samples, fecal samples, and sputum samples.

78-80. (canceled)

81. The method of claim 63, further comprising the step of detecting said expression of said first reporter molecule, wherein said expressed first reporter molecule is adapted to generate a detectable signal.

82. The method of claim 81, wherein said detectable signal is selected from the group consisting of an electrical signal, a chemical signal, an optical signal, and a detectable affinity to a second molecule.

83. (canceled)

84. A method of detecting the presence of drug resistance of a target bacteria in a sample to a drug of interest, comprising the steps of:
contacting said sample with a recombinant bacteriophage specific to said target bacteria comprising:
an exogenous nucleic acid sequence encoding a first reporter molecule that is flanked by a first flanking nucleic acid sequence comprising a nucleic acid sequence homologous with a first target nucleic acid sequence within the at least one target bacterium, wherein said first flanking nucleic acid sequences is homologous to a portion of a nucleic acid sequence that encodes a phenotype of said drug resistance of said target bacteria to said drug of interest;
assaying said sample for expression of said first reporter molecule, wherein expression of said first reporter molecule is indicative of said drug resistance of said target bacteria to said drug of interest.

85. The method of claim 84, wherein said sample is selected from the group consisting of environmental samples, plant samples, veterinary samples, food samples, livestock samples, and medical samples.

86. The method of claim 84, wherein said sample is selected from the group consisting of soil samples, water samples, vegetable samples, meat samples, blood samples, urine samples, tissue biopsy samples, mucus samples, fecal samples, and sputum samples.

87. The method of claim 84, further comprising the step of detecting said expression of said first reporter molecule, wherein said expressed first reporter molecule is adapted to generate a detectable signal.

88. The method of claim 87, wherein said detectable signal is selected from the group consisting of an electrical signal, a chemical signal, an optical signal, and a detectable affinity to a second molecule.
